(19) United States
(12) Patent Application Publication
(10) Pub. No.: US 2010/0168656 A1
(43) Pub. Date: Jul. 1, 2010

(54) APPARATUS AND METHODS FOR TREATING URETHRAL INCONTINENCE

(76) Inventors: Anthony Lee, Los Altos, CA (US); Hank Wuh, Honolulu, HI (US)

Correspondence Address: PATTERSON & SHERIDAN, L.L.P., 3040 POST OAK BOULEVARD, SUITE 1500, HOUSTON, TX, 77056, US

(21) Appl. No.: 12/088,654

(22) PCT Filed: Sep. 28, 2006
(86) PCT No.: PCT/US06/37666

§ 371 Date: Feb. 26, 2009

Related U.S. Application Data
(60) Provisional application No. 60/721,403, filed on Sep. 28, 2005.

(51) Int. Cl. A61M 25/10; A61M 5/00

(52) U.S. Cl. 604/36; 604/103.03; 604/98.02; 604/509

(57) ABSTRACT

The invention relates to apparatus (A) for injection of therapeutic agents into surrounding lumenal tissue, for example, to treat urinary incontinence and gastroesophageal reflux disease.

This patent application claims priority to U.S. patent application Ser. No. 60/721,403, filed Sep. 28, 2005, and is incorporated by reference herein as if set forth in its entirety.

### FIELD OF THE INVENTION

The present invention relates generally to medical devices and, more particularly, to an injection apparatus to target specific anatomy and facilitate the treatment of urinary incontinence and gastroesophageal reflux disease.

### BACKGROUND

One of the primary uses of the present invention is treatment of urinary incontinence. For example, many women suffer from incontinence caused by childbirth and/or obesity. Usually exercises to strengthen the pelvic floor muscles are the initial treatment for incontinence. If these exercises are ineffective, pharmacological solutions are usually attempted. As a final resort, open surgical repair of the bladder neck can be attempted. But surgical repair procedures are not successful in all patients and there is also risk associated with open surgical procedures, such as trauma, infection, and side effects of anesthesia.

As an alternative to surgical repair, urinary incontinence has been treated by injecting various substances into the tissue surrounding the urethra, i.e., the periurethral tissue, to add bulk to this tissue. The aim of this treatment is to compress the urethra at the level of the bladder neck to impede involuntary flow of urine from the bladder.

The use of sodium morrhuate for the treatment of stress incontinence has been reported (1). This material was not successful in treating incontinence, and pulmonary infarction was an observed complication. Paraffin and other sclerosing solutions have also been tried, also yielding poor results (2,3).

Polytetrafluoroethylene particles have been used as injectable balking material with a success rate from 30% to 86% in some studies (4,5,6,7,8,9). The complications associated with this procedure include foreign body granulomas that tended to migrate to distant organs, such as the lungs, liver, spleen, and brain (10).

Another injectable used recently is glutaraldehyde cross-linked bovine dermal collagen (Br. J. Urol., 75:359-363 (1995); Br. J. Urol., 75: 538-542 (1993)) (11). A major problem with the use of collagen was biodegradation with associated decrease in implant volume over time necessitating re-treatment (12) Collagen can also cause adverse immune responses and allergic reactions to bovine collagen have been described (13).

Other materials have been suggested for use in the treatment of vesicourectal reflux. These substances include polyvinyl alcohol foam, glass particles, a chondrocyte-alginate suspension, and a detachable silicone balloon (14,15,16,17).

The effectiveness of these bulking agents is often limited by the precision and circumferentialality of injection. If the injection is non-circumferential, it can produce an oblong lumen that does not adequately inhibit flow. If the injection is not made into the urinary sphincter, it will also not have the desired effect. Currently, in an effort to obtain circumferentiality and precision, time-consuming multiple injections are usually made.

Additionally, various injection apparatus and probes are described in U.S. Pat. Nos. 5,645,528; 6,090,063; 6,183,520; 6,666,848; 6,852,091; and 6,878,369; and U.S. Patent Publication Nos. 2005/0124852 and 2004/0037887; the entire disclosures of which are hereby incorporated herein by reference.

It is, therefore, an object of the present invention to provide apparatus and methods for effectively treating urinary incontinence and gastroesophageal reflux disease, with minimal to no complications.

### SUMMARY OF THE INVENTION

Generally, the invention relates to apparatus and methods for accurately introducing bulking agents into tissue surrounding bodily lumens, in particular a catheter for urethral incontinence (CUI) that precisely and consistently delivers a bulking agent to the urethral sphincter for the treatment of incontinence. The CUI utilizes a balloon to precisely position injection needles into the urethral sphincter and surrounding tissue without complicated and invasive visualization tools. More importantly, the plurality of injection needles in the present invention creates a longer and more circular treatment area within the bodily lumen for the injection of the bulking agent, improving treatment of the effected area over the prior art methods.

In one aspect, the invention relates to a medical delivery device, such as a catheter, that includes an elongate body defining at least one lumen and a deployable injection assembly disposed at a distal portion of the elongate body.

In various embodiments, the elongate body can define a plurality of lumens and may include a balloon or other securing means disposed proximate the distal end of the elongate body. The plurality of lumens can be used to deliver a therapeutic agent to the body or provide drainage of bodily fluids. At least one of the lumens could be disposed about a central longitudinal axis of the elongate body. The additional lumens could be disposed radially about the central longitudinal axis of the elongate body, or in essentially an orientation as necessary to suit a particular purpose. Additionally, one or more lumens could be in fluid communication with the balloon to inflate/deflate the balloon. Further, a central lumen of the catheter can be used as a conduit to drain the contents of the bladder. The device could also include valving to isolate the lumens or regulate the flow of materials therethrough. Alternatively or additionally, the device could include other structures to position and/or secure the device in place such as, for example, a molly bolt, a metallic fan, or a malecot.

The deployable injection assembly can include a plurality of hypodermic tubes disposed about the elongate body to deliver the therapeutic materials to the body. The tubes can have a retracted position, for example recessed within the elongate body to prevent the tubes from interfering with insertion of the device, and an extended position, where a sharpened end of the tubes extend beyond the outer surface of the elongate body and into the surrounding tissue.

The tubes can be disposed about the circumference of the elongate body and can be oriented radially with respect to the central longitudinal axis. The tubes can be disposed in multiple rows along the length of the elongate body, where the orientation of the rows of tubes may be aligned, offset, or helically arranged. The use of multiple rows of needles facilitates injecting the agent over a longer section of the bodily lumen, i.e., urethra, bladder neck, or esophagus, and not just, for example, the urethral sphincter. The distal ends of the needles may be modified to decrease the penetration force required.

In another aspect, the invention relates to a method of treating urinary incontinence or gastroesophageal reflux disease (GERD). The method includes the steps of providing a medical delivery device including an elongate body defining at least one lumen and a deployable injection assembly disposed at a distal portion of the elongate body; positioning the device within the body; deploying the injection assembly; and delivering a therapeutic agent to the surrounding tissue. The method also includes retracting the injection assembly and removing the device. The method may also include repositioning the device between multiple deployments of the injection assembly and delivering of therapeutic agents, e.g., a bulking agent. In one embodiment, the bulking agent can be gently warmed prior to injection to decrease viscosity. Decreased viscosity will reduce the force required to push the bulking agent through the injection assembly.

These and other objects, along with advantages and features of the present invention herein disclosed, will become apparent through reference to the following description and the accompanying drawings. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. In addition, the drawings are not necessarily to scale, emphasis instead generally being placed upon illustrating the principles of the invention. In the following description, various embodiments of the present invention are described with reference to the following drawings, in which:

FIG. 1 is a schematic plan view of a medical delivery device in accordance with one embodiment of the invention;

FIG. 2 is a schematic cross-sectional view of the device of FIG. 1 taken along line 2-2 in FIG. 1;

FIG. 3A is a schematic perspective view of an injection assembly portion of a medical delivery device in accordance with one embodiment of the invention;

FIG. 3B is a schematic plan view of a portion of a medical delivery device and injection assembly in accordance with one embodiment of the invention;

FIG. 4 is a schematic plan view of a medical delivery device with the injection assembly retracted in accordance with one embodiment of the invention;

FIGS. 5A-5B are schematic representations of one embodiment of a medical delivery device in operation in accordance with one embodiment of the invention;

FIGS. 6A-6B are schematic perspective views of an injection assembly in accordance with an alternative embodiment of the invention in a partial deployment and a full deployment, respectively;

FIG. 7 is a schematic representation of a medical delivery device deployed in accordance with one embodiment of the invention;

FIGS. 8A-8B are schematic perspective views of an alternative embodiment of an injection assembly in accordance with the invention; and

FIG. 9 is a schematic representation of an alternative embodiment of a medical delivery device in accordance with the invention.

### DETAILED DESCRIPTION

Embodiments of the present invention are described below. It is, however, expressly noted that the present invention is not limited to these embodiments, but rather the intention is that variations, modifications, and equivalents that are apparent to the person skilled in the art are also included.

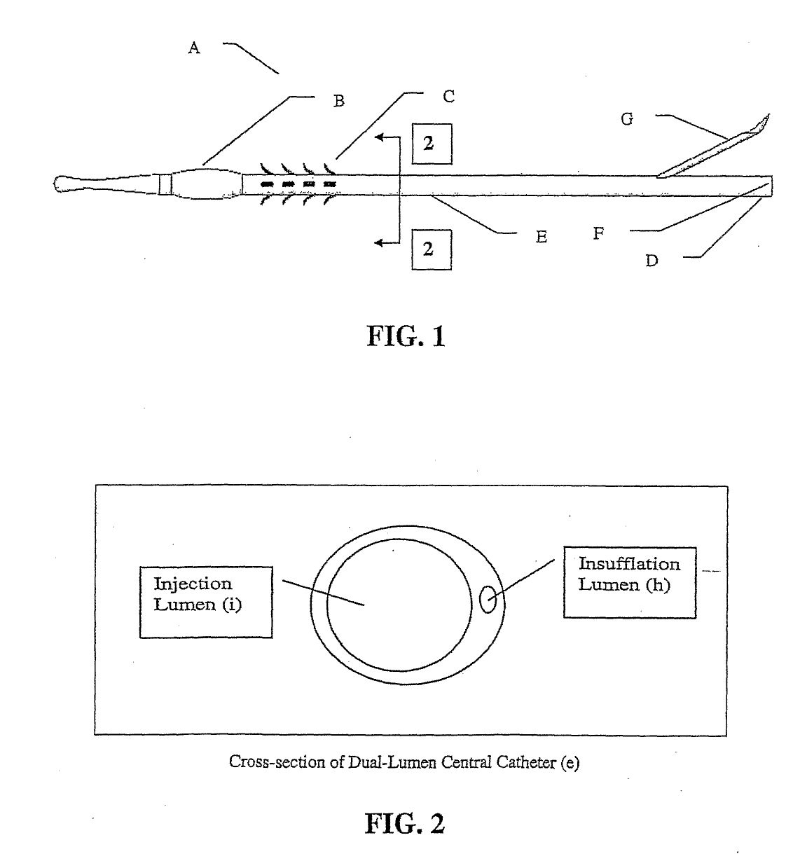

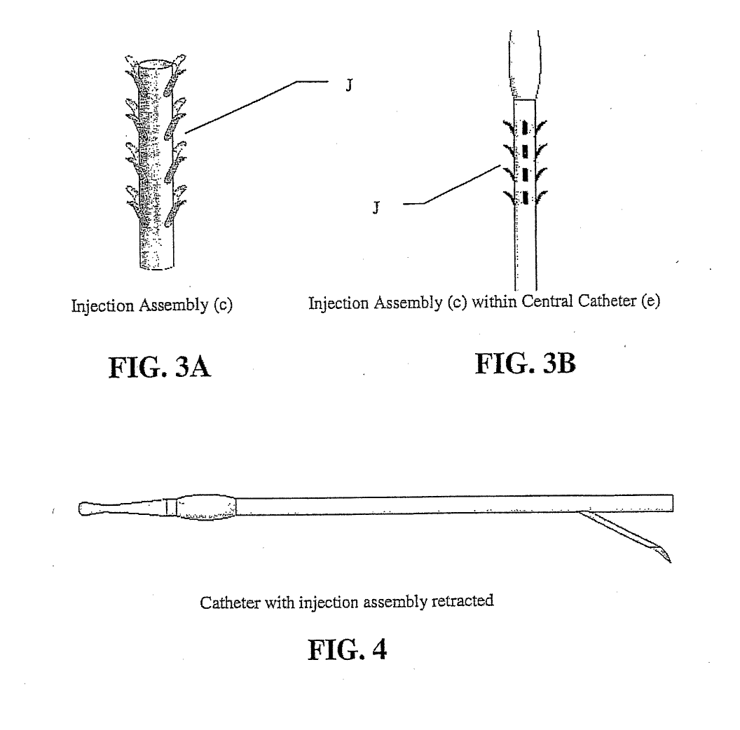

As shown in FIGS. 1-4, the device (A) includes a catheter (E), an injection assembly (C), and a positioning balloon (B) located at the distal end of the catheter. The balloon communicates with a proximal insufflation port (G) via the insufflation lumen (H) of the dual lumen central catheter (E). The balloon can be inflated and/or deflated through this insufflation port (G).

Just proximal to the positioning balloon is the injection assembly (C). The injection assembly lies within the central catheter (E) and contains a plurality of sharpened hypodermic tubes (J) arranged circumferentially around the catheter with the sharpened end pointed outward. In a particular embodiment, there are twelve needles; however, the number and arrangement of the needles will vary to suit a particular application.

The proximal ends of the hypodermic needles are mechanically coupled to a deployment assembly (D) via, for example, a linkage or push wire. Moving the deployment assembly distally will cause the needles to protrude from the catheter. Moving the deployment assembly proximally retracts the needles back into the catheter. The lumens of the needles communicate directly with an injection port (F) via the central lumen of the catheter, allowing the user to inject materials from the injection port through the central lumen of the catheter and out the needles.

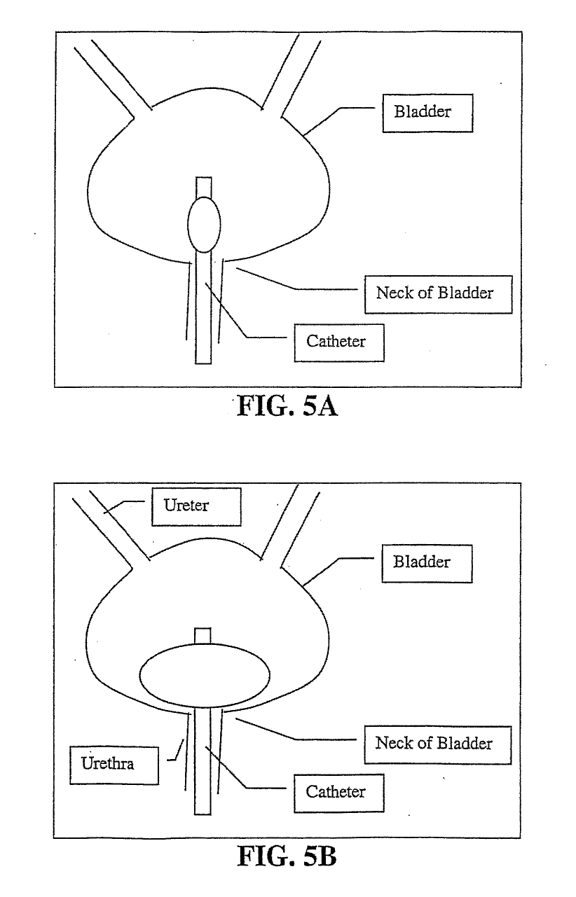

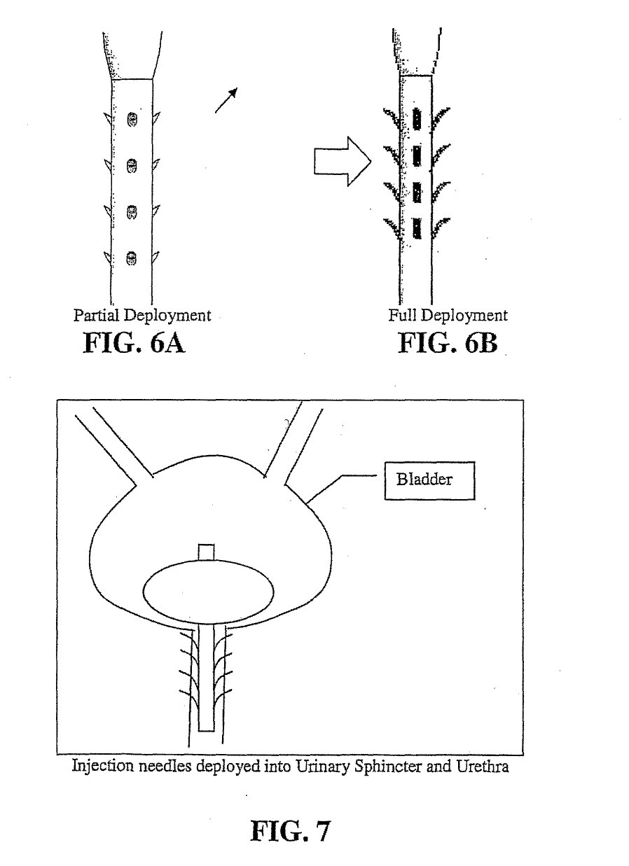

In one possible application of use (see FIGS. 5-7), the device is inserted through the urethra until the distal end reaches the bladder. Once the positioning balloon is in the bladder, it is inflated. The catheter is then pulled down until the positioning balloon rest firmly against the neck of the bladder. This action ensures that the injection needles will be deployed directly into the neck of the bladder without the need for direct surgical vision.

Once the catheter is in proper position, the injection assembly is pushed forward, forcing the needles to deploy outwardly and distally into the urinary sphincter (located at the neck of the bladder) and smooth muscle layer of the urethra. A bulking agent is then injected through the needles into the urinary sphincter and urethra. The number of needles and their position in the device of the present invention, create a longer, more circumferential treatment area within the bodily lumen for the injection of the bulking agent, improving treatment of the effected area over the prior art. The needles are then withdrawn back into the catheter. The positioning balloon is deflated and the catheter is removed from the patient.

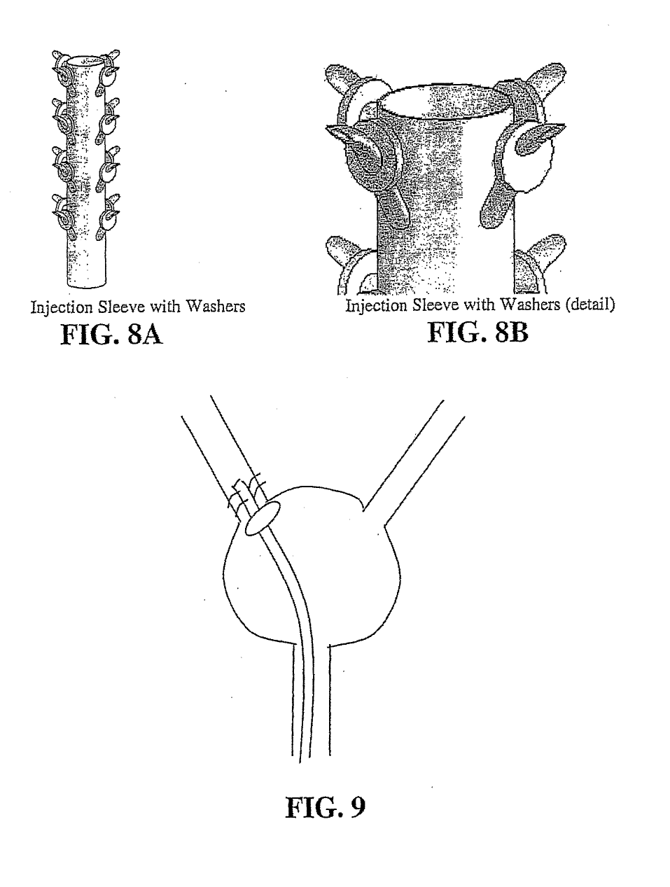

In one alternative embodiment, as shown in FIGS. 8A and 8B, washers can be placed toward the distal end of the injection needles. The washers will control needle penetration, thus insuring that the bulking agent is injected into the smooth muscle layer of the urethra.

In an alternative method of use, the device is used to treat GERD. A simple modification in the diameter of the catheter for urinary incontinence can be made to treat GERD. The material may be injected into the wall of the esophagus to thicken the wall and narrow the gastroesophageal junction into the stomach. During proper operation of the lower esophageal sphincter, the lower esophageal sphincter opens to allow food to pass into the stomach and closes to prevent food and acidic stomach fluids from flowing back up into the esophagus. Gastroesophageal reflux occurs when the lower esophageal sphincter is weak or relaxes inappropriately, allowing the stomach's contents to retrograde or flow up into the esophagus.

This retrograde flow of gastric contents back into the esophagus, through what should be a one-way valve into the stomach, can damage the esophagus. More particularly, the contents of the stomach are very acidic; and the lining of the stomach is specially designed to cope with the lower pH contents. The esophagus, on the other hand, is not suited for such exposure to highly acidic materials. Thus, when acid retrogrades from the stomach into the esophageal tissues, irritation and inflammation will often result to these tissues.

Additional uses for the device include treating ureter reflux (see FIG. 9). Approximately 1-2% of infants suffer from ureter reflux. In this condition, the one way valve between the ureter and the bladder does not close completely, allowing urine to back up into the kidneys. Surgical intervention is a common treatment for the malady. The urethral catheter could be used to treat ureter reflux. The design would be modified so that the deployment needles are placed distal to the positioning balloon.

Furthermore, the device can be used for performing prostate injection. A pair of special needles are placed 180 degrees apart on the circumference of the catheter. These needles will be longer than the other needles of the injection assembly.

The invention also relates to other methods of injecting bulking agents, such as needle-less injection, injection of anesthesia first, injection by expansion, and injection by a screw mechanism. With needle-less injection, the bulking agent is injected under high pressure. Needle-less injection requires the bulking agent to be nebulized into small particles. These small particles are forced at high pressure between the cells of the inner walls of the urethra.

Prior to deploying the needles, a surface anesthesia can be sprayed onto the inside wall of the urethra or other bodily lumen. This will reduce the pain associated with the insertion of the needles.

Injection by expansion involves the needles being attached to an inflatable member, for example a balloon. In order to deploy the needles, the balloon is inflated, driving the needles outwardly, similar to a porcupine configuration. Additionally, a worm screw mechanism can be used for deploying/retracting the positioning device and/or the needles.

The shape and dimensions of the device will vary to suit a particular application; for example, a longer catheter may be required to treat ureter reflux as opposed to treating uretheral incontinence. Also, the size and shape of the device may vary as necessary depending on the physiology of the patient to be treated; for example, a smaller device would likely be used for treating a child as opposed to an adult. The device is typically manufactured of biocompatible materials, such as polymers, for example polyurethanes, silicones, polyethylenes, nylons, polyesters, and polyester elastomers.

The various devices described herein may be manufactured by, for example, injection molding or modifying an extruded tube. For example, extrusion may be used to provide a uniform polymeric tube, to which a hub is attached at one end and the other end is sealed. Insert molding or a subsequent mechanical operation can then be used to provide the desired geometry of other components. The other components, for example the positioning balloon, can be bonded to the catheter.

Having described certain embodiments of the invention, it will be apparent to those of ordinary skill in the art that other embodiments incorporating the concepts disclosed herein may be used without departing from the spirit and scope of the invention. The described embodiments are to be considered in all respects as only illustrative and not restrictive.

### REFERENCES

1) J. Obstet. Gynaecol., 45:67-71(1938)
2) Acta Urol. Belg., 23:259-262(1955)
3) Urol. Int., 15:225-244(1963)
4) J. Urol., 111:180-183(1974)
5) Br. J. Urol., 55:208-210(1983)
6) BMJ 228;192 (1984)
7) J. Urol., (Paris), 62:39-41(1987)
8) Br. J. Urol., 62:39-41 (1988)
9) Aust. N. Z. J. Surg., 61:663-666 (1966)
10) JAMA, 251:3227-3281 (1984)
11) Med. J. Aust., 158:89-91 (1993)
12) J. Urol., 150:745-747 (1993)
13) Br. J. Urol., 75:359-363 (1995)
14) J. Urol., 144:531-533 (1990)
15) J. Urol., 148:645 (1992)
16) J. Urol., 150:745-747 (1993)
17) J. Urol., 148:724-728 (1992)

1. A medical device for injection of fluids into tissue surrounding a bodily lumen of a patient, comprising an elongate body being a generally tubular shaft having a first end, a second end, and an outer surface and at least one lumen extending between the first end and the second end of the shaft defining at least two lumens, wherein
at least one of said lumens are disposed about the central longitudinal axis of said elongate body;
a tip mounted on the second end of the shaft, the tip comprising a monolithic member including an inflatable balloon portion or other securing means and a tip portion extending from the balloon portion and configured to facilitate insertion of the catheter within a urethra, the balloon portion being arranged and configured to slip fit over and be secured on the shaft second end, thereby securing the tip portion to the shaft a balloon or other securing means disposed proximate to the distal end of the elongate body;
at least one of the remaining lumens being in fluid communication with the balloon or securing means capable of causing the balloon to inflate or deflate;
said device optionally comprising additional lumens being connected to a deployable injection assembly disposed about circumferentially along the central longitudinal axis of the elongate body, at a distal portion of the elongate body and disposed proximate to the central longitudinal axis of the elongate body; and
when said device includes a deployable injection assembly, at least one of the plurality of lumens in said assembly being capable of delivering a therapeutic agent to said tissues surrounding bodily lumens.

2. The medical device of claim 1, wherein said securing means for positioning and/or securing the device in place includes a molly bolt, a metallic fan, or a malecot.

3. The medical device of claim 1, wherein said elongate body comprises a multiple lumen central catheter (E), an injection assembly (C), and a positioning balloon (B) located at the distal end of the catheter, said balloon (B) being in communication with a proximal insufflation port (G) via an insufflation lumen (H) of the dual lumen central catheter (E), allowing the balloon to be inflated and/or deflated through this insufflation port (G) by increasing or decreasing the fluid pressure within the insufflation lumen (H) by a pump or bulb means;
said injection assembly (C) being proximal to the positioning balloon and positioned within the central catheter (E) and containing a plurality of hypodermic tubes (J) having a first end and a second end and a generally tubular shaft, wherein said first end is sharpened and at the distal end of the catheter and arranged circumferentially around the central catheter lumen as a single ring formation with their sharpened ends pointed outward away from the central catheter lumen;
the proximal ends of the hypodermic tubes (J) are mechanically connectively attached to a deployment assembly (D) by a linkage or push wire means such that slidably moving the deployment assembly distally causes the sharpened first ends of the hypodermic tubes (J) to protrude out and away from the catheter lumen, and slidably moving the deployment assembly proximally cause the sharpened first ends of the hypodermic tubes (J) toward the central lumen and retract into the catheter; and
said lumens of the hypodermic tubes (J) communicate directly with an injection port (F) via the lumens of the catheter, which is capable of delivering materials from the injection port (F) through the central lumen of the catheter and out the sharpened first ends of the hypodermic tubes (J) to deliver materials to the tissues surrounding the device.

4. A medical device for injection of fluids into tissue surrounding a bodily lumen of a patient, comprising an elongate body having a multiple lumen central catheter (E), an injection assembly (C), and a positioning balloon (B) located at the distal end of the catheter, said balloon (B) being in communication with a proximal insufflation port (G) via an insufflation lumen (H) of the dual lumen central catheter (E), allowing the balloon to be inflated and/or deflated through this insufflation port (G) by increasing or decreasing the fluid pressure within the insufflation lumen (H) by a pump or bulb means;
said injection assembly (C) being proximal to the positioning balloon and positioned within the central catheter (E) and containing a plurality of hypodermic tubes (J) having a first end and a second end and a generally tubular shaft, wherein said first end is sharpened and at the distal end of the catheter and arranged circumferentially and axially around the central catheter lumen as a plurality of ring formations with their sharpened ends pointed outward away from the central catheter lumen;
the proximal ends of the hypodermic tubes (J) are mechanically connectively attached to a deployment assembly (D) by a linkage or push wire means such that slidably moving the deployment assembly distally causes the sharpened first ends of the hypodermic tubes (J) to protrude out and away from the catheter lumen, and slidably moving the deployment assembly proximally cause the sharpened first ends of the hypodermic tubes (J) toward the central lumen and retract into the catheter; and
said lumens of the hypodermic tubes (J) communicate directly with an injection port (F) via the central lumen of the catheter, which is capable of delivering materials from the injection port (F) through the central lumen of the catheter and out the sharpened first ends of the hypodermic tubes (J) to deliver materials to the tissues surrounding the device.

5. A medical device for injection of fluids into tissue surrounding a bodily lumen of a patient, comprising an elongate body having a multiple lumen central catheter (E), an injection assembly (C), and a positioning balloon (B) located at the distal end of the catheter, said balloon (B) being in communication with a proximal insufflation port (G) via an insufflation lumen (H) of the dual lumen central catheter (E), allowing the balloon to be inflated and/or deflated through this insufflation port (G) by increasing or decreasing the fluid pressure within the insufflation lumen (H) by a pump or bulb means;
said injection assembly (C) being proximal to the positioning balloon and positioned within the central catheter (E) and containing a plurality of hypodermic tubes (J) having a first end and a second end and a generally tubular shaft, wherein said first end is sharpened and at the distal end of the catheter and arranged circumferentially and axially around the central catheter lumen as a plurality of ring formations with their sharpened ends pointed outward away from the central catheter lumen;
the proximal ends of the hypodermic tubes (J) are mechanically connectively attached to a deployment assembly (D) by a linkage or push wire means such that slidably moving the deployment assembly distally causes the sharpened first ends of the hypodermic tubes (J) to protrude out and away from the catheter lumen, and slidably moving the deployment assembly proximally cause the sharpened first ends of the hypodermic tubes (J) toward the central lumen and retract into the catheter;
said first ends of plurality of hypodermic tubes (J) also having a ring of greater width disposed circumferentially around each hypodermic tube (J) and outside of the outer surface and positioned a fixed distance away from the sharpened first end of said hypodermic tube (J) so that the ring limits the penetration of the sharpened first end of the hypodermic tube into the surrounding tissues when the deployment assembly is moved distally; and
said lumens of the hypodermic tubes (J) communicate directly with an injection port (F) via the central lumen of the catheter, which is capable of delivering materials from the injection port (F) through the central lumen of the catheter and out the sharpened first ends of the hypodermic tubes (J) to deliver materials to the tissues surrounding the device.

6. A method for delivering a therapeutic agent to a bladder tissue in a human, to treat incontinence, which method comprises:
a) inserting the device of claim 1 through the urethra until the distal end reaches the bladder;
b) positioning the balloon of the device of claim in the bladder, and inflating said balloon sufficiently such that the device cannot be moved back down the urethra;
c) pulling the catheter down the urethra until the positioning balloon rest firmly against the neck of the bladder, indicating that the catheter is in the correct position;
d) moving the injection assembly distally, forcing the hypodermic tubes of the device to deploy outwardly and distally into the surrounding tissue and smooth muscle layer;
e) injecting a therapeutic agent through the hypodermic tubes of the device into the urinary sphincter in a circumferential manner and urethra in a sufficient quantity to achieve the clinical effect;
f) moving the injection assembly proximally, moving the hypodermic tubes of the device toward the central lumen and retracting into the catheter; and
g) deflating the balloon of said device and removing the device from the urethra.

7. A method for delivering a therapeutic agent to esophageal sphincter tissue in a human, to treat reflux disease, which method comprises:
a) inserting the device of claim 1 through the esophagus until the distal end reaches the stomach;
b) positioning the balloon of the device of claim in the stomach, and inflating said balloon sufficiently such that the device cannot be moved back up the esophagus;
c) pulling the catheter up the esophagus until the positioning balloon rest firmly against the neck of the stomach, indicating that the catheter is in position;
d) moving the injection assembly distally, forcing the hypodermic tubes of the device to deploy outwardly and distally into the surrounding tissue and smooth muscle layer;
e) injecting a therapeutic agent through the hypodermic tubes of the device into the esophageal sphincter in a circumferential manner and in a sufficient quantity to achieve the clinical effect;
f) moving the injection assembly proximally, moving the hypodermic tubes of the device toward the central lumen and retracting into the catheter; and
g) deflating the balloon of said device and removing the device from the stomach.

8. The medical device of claim 5, wherein said first ends of plurality of hypodermic tubes (J) having a barb or spur instead of a ring of greater width disposed circumferentially around each hypodermic tube (J), said barb or spur positioned a fixed distance away from the sharpened first end of said hypodermic tube (J) so that the barb or spur limits the penetration of the sharpened first end of the hypodermic tube into the surrounding tissues when the deployment assembly is moved distally

9. The medical device of claim 1, wherein said lumen disposed about the central longitudinal axis of said elongate body also being capable of providing drainage of bodily fluids from said bodily lumens of a patient.
